(19) United States
(12) Patent Application Publication
(10) Pub. No.: US 2018/0368919 A1
(43) Pub. Date: Dec. 27, 2018

(54) MEDICAL COORDINATE MEASURING DEVICE AND MEDICAL COORDINATE MEASURING METHOD

(71) Applicant: Aesculap AG, Tuttlingen (DE)

(72) Inventors: Tobias Pfeifer, Waldmoessingen (DE); Holger Broers, Uplengen-Spols (DE)

(21) Appl. No.: 15/982,157

(22) Filed: May 17, 2018

Related U.S. Application Data
(63) Continuation of application No. PCT/EP2016/075801, filed on Oct. 26, 2016.

(30) Foreign Application Priority Data
Nov. 19, 2015 (DE) 102015120068.6
May 19, 2016 (DE) 102016109173.1

(51) Int. Cl. A61B 34/20; A61B 90/00; G01C 3/10

(52) U.S. Cl. CPC A61B 34/20; A61B 90/37; G01C 3/10; A61B 90/39; A61B 2034/2055; G01B 11/245; G01B 2210/52; A61B 2090/371; A61B 2034/2072; A61B 2090/3937; A61B 2034/2068

(57) ABSTRACT

A medical radiation-based coordinate measuring device is provided which comprises a first image sensor unit and a second image sensor unit for providing a first luminosity characteristics data set and a second luminosity characteristics data set. A data processing unit is provided for determining the object coordinates of object points in the space, which are imaged by means of electromagnetic radiation, by triangulation of the luminosity characteristics data sets. At least one spacing measuring unit for the object points is provided for providing at least one spacing data set. The data processing unit is configured and programmed in such a way that, before the triangulation, it adds a spacing information to at least the first luminosity characteristics data set on the basis of a spacing data set. In addition, a coordinate measuring method is provided.

### CROSS-REFERENCE TO RELATED APPLICATIONS

This application is a continuation of international application number PCT/EP2016/075801, filed on Oct. 26, 2016, and claims the benefit of German application number DE 10 2015 120 068.6, filed Nov. 19, 2015, and German application number DE 10 2016 109 173.1, filed May 19, 2016, which are incorporated herein by reference in their entirety and for all purposes.

### FIELD OF THE INVENTION

The present invention relates to a medical radiation-based coordinate measuring device, comprising a first image sensor unit and a second image sensor unit for providing a first luminosity characteristics data set and a second luminosity characteristics data set, respectively, and a data processing unit for determining the object coordinates of object points in the space, which are imaged by means of electromagnetic radiation, by triangulation of the luminosity characteristics data sets.

In addition, the invention relates to a medical radiation-based coordinate measuring method using a coordinate measuring device of that kind.

### BACKGROUND OF THE INVENTION

A coordinate measuring device of the kind described hereinabove is used for example during a navigation-supported surgical procedure. Hereby, the goal is to determine the coordinates (position) and the orientation of object points to each other. Changes in the coordinates and the orientation may be determined with respect to time and the object points in the space may thereby be tracked. It is known to define a reference coordinate system by means of a surgical marking apparatus that is fixed to a patient. For this purpose, the marking apparatus has self-luminous or reflective marking elements that can be detected and tracked particularly well by the coordinate measuring device. Tracking methods of this kind prove themselves in practice, but the requirements on the imaging of the marking elements are not inconsiderable, and there is a restriction to a relatively small number of object points. Moreover, there are reservations on the part of the patients or surgeons against the use of marking apparatuses, because these usually have to be invasively fixed to a bone of the patient. However, there exist proposals for the design of non-invasive marking apparatuses, as are described in DE 10 2013 112 375 A1 or in DE 10 2014 104 800 A1, for example.

It would be desirable for a multitude of object points, in a sense a flat object, to be able to be tracked simultaneously in a medical coordinate measuring method, in order to increase the information content of the tracking method and to forgo a separate marking apparatus, if possible.

In conventional medical coordinate measuring devices having a stereo camera system, a respective direction to object points can be determined using a respective luminosity characteristics data set comprising a luminosity or intensity information. In the triangulation, the two directions are overlaid by a so-called “spatial intersection”, in order to ascertain the position of the object point in the space. Even if the image sensor units possess a comparatively high resolution, ambiguities in the reading of the position of the object points may hereby arise (so-called “ghost points”). Also for this reason, the use of stereo coordinate measuring devices without the use of marking apparatuses is restricted, which have redundant marking elements to address the aforementioned problem.

An object underlying the present invention is to provide a generic coordinate measuring device and a coordinate measuring method with which a more reliable determination of the object coordinates is possible.

### SUMMARY OF THE INVENTION

In a first aspect of the invention, a medical radiation-based coordinate measuring device comprises a first image sensor unit and a second image sensor unit for providing a first luminosity characteristics data set and a second luminosity characteristics data set, respectively, and a data processing unit for determining the object coordinates of object points in the space, which are imaged by means of electromagnetic radiation, by triangulation of the luminosity characteristics data sets. The coordinate measuring device comprises at least one spacing measuring unit for the object points for providing at least one spacing data set, and the data processing unit is configured and programmed in such a way that it, before the triangulation, adds a spacing information to at least the first luminosity characteristics data set on the basis of a spacing data set.

In a second aspect of the invention, in a coordinate measuring method a first luminosity characteristics data set and a second luminosity characteristics data set, respectively, are provided with a first image sensor unit and a second image sensor unit, and object coordinates of object points in the space, which are imaged by means of electromagnetic radiation, are determined with a data processing unit by triangulation of the luminosity characteristics data sets. A spacing data set is provided with at least one spacing measuring unit, and the data processing unit, before the triangulation, adds a spacing information to at least the first image data set on the basis of the spacing data set.

### BRIEF DESCRIPTION OF THE DRAWINGS

The foregoing summary and the following description may be better understood in conjunction with the drawing figures, of which:

FIG. 1: shows a schematic depiction of a coordinate measuring device in accordance with the invention;

FIG. 2: shows a schematic depiction of a further coordinate measuring device in accordance with the invention;

FIG. 3: shows a schematic depiction of a third coordinate measuring device in accordance with the invention; and

FIG. 4: shows a cluster triangulation using five image sensor units for calibrating a coordinate measuring device.

### DETAILED DESCRIPTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a medical radiation-based coordinate measuring device, comprising a first image sensor unit and a second image sensor unit for providing a first luminosity characteristics data set and a second luminosity characteristics data set, respectively, and a data processing unit for determining the object coordinates of object points in the space, which are imaged by means of electromagnetic radiation, by triangulation of the luminosity characteristics data sets. The coordinate measuring device comprises at least one spacing measuring unit for the object points for providing at least one spacing data set, and the data processing unit is configured and programmed in such a way that it, before the triangulation, adds a spacing information to at least the first luminosity characteristics data set on the basis of a spacing data set.

It is taken into account in the invention that ambiguities in the determination of the coordinates of the object points may be avoided and the accuracy increased if, in addition to the luminosity characteristics data sets, further information is provided. For this purpose, at least one spacing data set is provided by means of at least one spacing measuring unit. Using the at least one spacing data set, the data processing unit may supplement at least the first luminosity characteristics data set and may expand it by a spatial depth information. The original first luminosity characteristics data set contains with high accuracy an indication of the respective direction of object points commencing from the first image sensor unit. Due to the additional spacing information from the at least one spacing data set, the position of the object point may already be approximately determined. In particular, ambiguities can be ruled out before they occur. In addition to the information on the direction of the object points, a spacing information is available, i.e. for each object point a direction vector and an information on its length. In a subsequent triangulation, with use of the coordinate measuring device in accordance with the invention, it results in a higher accuracy in the determination of the position of the object points than in the conventional coordinate measuring device. Corresponding to the preceding considerations, the first luminosity characteristics data set allows for avoiding possible limitations in the validity of the spacing data set and for misidentifications of the object points to be ruled out before they arise. In a spacing measurement, limitations with measuring errors occurring therefrom in the case of edge profiles may arise, which may be addressed through additional information from the first luminosity characteristics data set.

Just as the image sensor units are in defined spatial relationship to each other, the at least one spacing unit may preferably be positioned in defined spatial relationship to the image sensor units. Using the relative orientation, it is possible for the data processing unit to associate image regions in the luminosity characteristics data sets to each other and to associate image regions in the luminosity data sets to a region in the spacing data set, respectively.

Advantageously, the data processing unit is configured and programmed in such a way that it, before the triangulation, adds a spacing information to the second luminosity characteristics data set on the basis of a spacing data set. Correspondingly like in the first luminosity characteristics data set, the data processing unit may expand the second luminosity characteristics data set by the additional spacing information. For the object points, respective direction vectors and information on their lengths are available, based on a respective luminosity characteristics data set, expanded by a spacing information. The accuracy of the determination of the position of the object points may thereby be increased.

Alternatively, provision may be made for the data processing unit to be configured and programmed in such a way that it triangulates the first luminosity characteristics data set, which is supplemented by the spacing information, with the second luminosity characteristics data set.

It is advantageous if the at least one spacing measuring unit is or comprises a time-of-flight (TOF)-measuring unit that generates the spacing data set using a light propagation time method. The TOF-measuring unit comprises for example a PMD-sensor (photonic mixing device) that provides at least one spacing information about a spacing data set. The spacing information comes from a propagation time measurement for light that is used to illuminate the object points, is reflected thereby, and is detected by the PMD-sensor. The distance of an object point to the PMD-sensor is proportional to the propagation time of the light, such that the spacing information may be generated using the propagation time.

The TOF-measuring unit may, in particular via the PMD-sensor, provide a luminosity characteristics data set in addition to the spacing data set. This will subsequently be discussed.

The time-of-flight (TOF)-measuring unit preferably has an illumination unit for illuminating the object points with light of a spectral range that differs from a spectral range to which the image sensor units are sensitive. By means of the illumination unit, light of a certain spectral range may be emitted that is detected by the PMD-sensor after reflection on an object. The use of a different spectral range in the image sensor units offers the advantage that TOF-measuring unit is not disrupted by a possible illumination for the image sensor units and that these are not disrupted by the light from the illumination unit. The accuracy of the measurements in the image data sets and in the spacing data set may thereby be increased.

Provision may be made for a spacing measuring unit, which is spatially separated from the image sensor units, to be provided, in particular a time-of-flight (TOF)-measuring unit. In a device of this kind, provision may be made for the image sensor units to be positioned in stereo arrangement and for a spatially separated spacing measuring unit and in particular TOF-measuring unit to be provided that provides a spacing data set.

It proves to be advantageous if the coordinate measuring device comprises at least one combined image sensor spacing measuring unit that forms an image sensor unit for providing a luminosity characteristics data set and a spacing measuring device for providing a spacing data set, in particular a time-of-flight (TOF)-measuring unit, and if the spacing information is added to the luminosity characteristics data set on the basis of the spacing characteristics data set. Correspondingly, a combined unit may be provided, which delivers both a luminosity characteristics data set as well as a spacing data set, in order to, before the triangulation, expand the luminosity characteristics data set by a spacing information. In this way, the coordinate measuring device may get by with merely two units, namely a combined image sensor spacing measuring unit and a further unit that provides at least one further luminosity characteristics data set is sufficient.

In an advantageous embodiment of the coordinate measuring device, provision is favorably made for it to comprise two combined image sensor spacing measuring units that form in particular time-of-flight (TOF)-measuring units, and for the respective spacing information to be added to the respective luminosity characteristics data set on the basis of the respective spacing data set. The triangulation may thereby be based on data sets of two spacing measuring units and in particular TOF-measuring units having a respective spacing data set and a respective luminosity characteristics data set. These are provided by PMD-sensors, for example. A separate spacing measuring unit may be thereby be dispensed with.

The data processing unit may be configured and programmed in such a way that it identifies the image points in the luminosity characteristics data set of the combined image sensor spacing measuring unit with image points in the luminosity characteristics data set of the second image sensor unit. Imaging of object points may thereby be recognized via a luminosity analysis of a respective luminosity characteristics data set of the image sensor units and of the luminosity characteristics data set of the spacing measuring unit, and may be associated with each other. This allows for increasing accuracy through redundant information, with which accuracy signal contributions in the luminosity characteristics data sets are provided with the spacing information coming from the at least one spacing data set.

The data processing unit is preferably configured and programmed in such a way that it processes the first luminosity characteristics data set, the second luminosity characteristics data set and/or the at least one spacing data set in real time, in order to determine in real time the position of the object points in the space.

Provision may be made for the first image sensor unit and the second image sensor unit to have a joint image sensor that has two image sensor regions that each deliver a luminosity characteristics data set. Each image sensor region may be associated with an optics of its own, by means of which the object points are imaged to the image sensor region.

In a corresponding manner, provision may be made for two spacing measuring units to have a joint image sensor that has two image sensor regions that each deliver a spacing data set. Each image sensor region may, in an exemplary embodiment, be associated with an optics of its own, by means of which the object points are imaged to the image sensor region.

Favorably, a resolution of optical sensors of the image sensor units and/or of spacing measuring units is identical.

It is advantageous if two or more image sensor units and/or two or more spacing measuring units are identically configured.

It is favorable if the image sensor units and/or the spacing measuring unit are sensitive in at least one of the following spectral ranges at least over a specifiable or specified wavelength range:
infrared;
visible light;
ultraviolet.

Advantageously, the coordinate measuring device comprises more than two image sensor units, wherein a luminosity characteristics data set is generable with a respective image sensor unit. A respective image data set may, before the triangulation by the data processing unit, be supplemented by the spacing information on the basis of the at least one spacing data set. Provision may hereby be made in particular for the more than two units to be combined image sensor spacing measuring units, in particular comprising a TOF-measuring unit.

It is advantageous if the coordinate measuring device comprises a storage unit that may be coupled to the data processing unit or be integrated therein. Attributes of observable object points are preferably stored in the storage unit, wherein the data processing unit is configured and programmed in such a way that it uses said attributes in the identification and tracking of the object points. Using the stored attributes, the data processing unit may more simply identify the object points in the data sets. This also facilitates the tracking of the object points. The attributes may, in a certain sense, be seen as a constraint on or necessary feature of the object points.

Examples of attributes of observable object points that are stored in the storage units are their relative position, their shape, their luminosity and/or their spectral sensitivity.

The attributes may relate to at least one of the following:
the anatomy of a patient;
a medical instrument;
an implant.

In the anatomy of the patient, the instrument and/or the implant, thanks to 3D-matching algorithms stored in the data processing unit and on the basis of the attributes in the storage unit, a tracking of the patient, the instrument, and/or the implant is preferably made possible, even if these are not provided with a marking apparatus.

The coordinate measuring device is favorably configured as or comprises a measuring system having a housing accommodating the image sensor units and, where applicable, at least one spacing measuring unit. The image sensor units and, where applicable, a spacing measuring unit are positioned in the housing in defined spatial and immovable relationship to each other. The housing may also accommodate the illumination unit of the at least one spacing measuring unit.

The data processing unit may be integrated into the housing.

Alternatively, provision may be made for the data processing unit to be positioned, at least partially, in a separated housing of the coordinate measuring device. The data exchange may be provided in a wireless or wired manner.

In an advantageous embodiment of a different kind, provision may be made for the image sensor units and, where applicable, the at least one spacing measuring unit to be freely positionable relative to each other. According to use case, it may be preferred, for example for achieving a higher accuracy, to freely position the image sensor units and, where applicable, at least one spacing measuring unit.

A calibration of the coordinate measuring device may be carried out using additional information for captured (test) objects. After arranging the image sensor units and, where applicable, the at least one spacing measuring unit, the luminosity characteristics data sets may be simultaneously processed, in particular by means of a cluster triangulation. Taking into account the additional information, the imaging characteristics of the coordinate measuring device are determined for later measurements.

The coordinate measuring device may be set up for extracorporeal and/or intracorporeal use (it may be configured as or comprise an exoscope, endoscope, a navigation camera, etc.)

As mentioned hereinabove, the present invention also relates to a medical radiation-based coordinate measuring method.

The object stated hereinabove is achieved by a coordinate measuring method in which, in accordance with the invention, a first luminosity characteristics data set and a second luminosity characteristics data set, respectively, are provided with a first image sensor unit and a second image sensor unit, and object coordinates of object points in the space, which are imaged by means of electromagnetic radiation, are determined with a data processing unit by triangulation of the luminosity characteristics data sets, wherein a spacing data set is provided with at least one spacing measuring unit, and the data processing unit, before the triangulation, adds a spacing information to at least the first image data set on the basis of the spacing data set.

The advantages already mentioned in conjunction with the description of the coordinate measuring device in accordance with the invention may also be achieved using the coordinate measuring method. To avoid repetition, reference may be made to the preceding statements.

It is advantageous if a time-of-flight (TOF)-measuring unit is used as a spacing measuring unit.

In particular, it is favorable if at least one combined image sensor spacing measuring unit is used that forms both an image sensor unit for providing a luminosity characteristics data set and a spacing measuring unit for providing a spacing data set, in particular a time-of-flight (TOF)-measuring unit.

Further advantageous embodiments of the coordinate measuring method in accordance with the invention arise from the attributes of advantageous embodiments of the coordinate measuring device in accordance with the invention, such that reference may be made to the preceding embodiments in this regard.

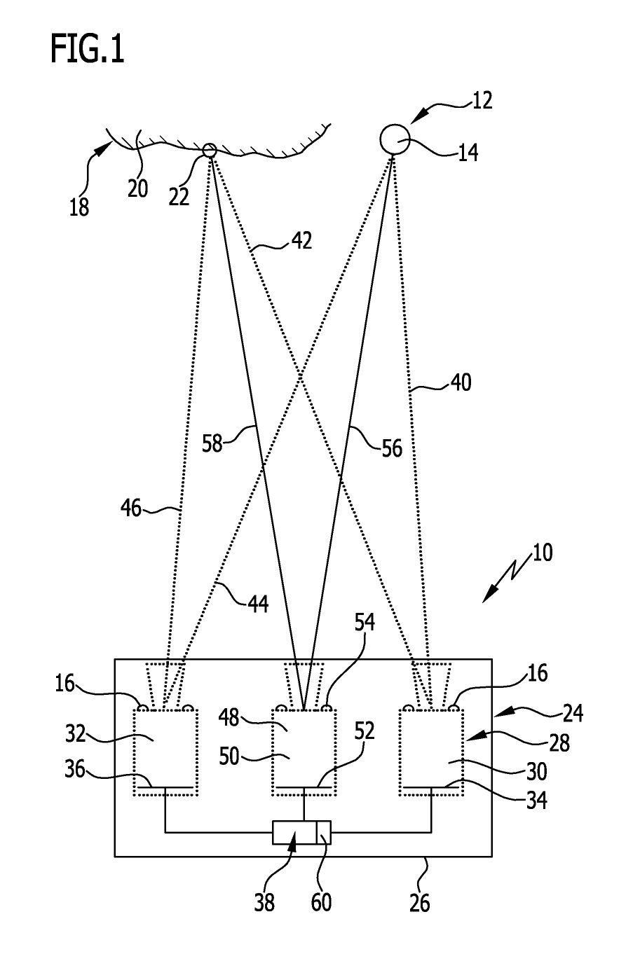

FIG. 1 shows in schematic depiction a medical radiation-based coordinate measuring device in accordance with the invention, which is attributed with the reference numeral 10, for carrying out a coordinate measuring method in accordance with the invention. The coordinate measuring device 10 may be used intraoperatively in order to image object points and track them in the space. For example, the drawing shows an object point 12 being a marking element 14 of surgical marking apparatus, the rest thereof not being depicted. The marking element 14 may be configured to be self-luminous or reflective to electromagnetic radiation that is emitted by a lighting apparatus 16 of the coordinate measuring device 10. The marking apparatus may for example, in a manner not-shown, which is known per se, be fixed to a body part of a patient and form a reference coordinate system on the body part.

A further object 18 to be detected and tracked comprises a multitude of individual object points. The object 18 is a body part 20 of a patient, for example an extremity. The drawing shows schematically how an exemplary, selected object point 22 is imaged with the coordinate measuring device 10.

For the subsequent embodiments, reference is made thereto that the further description is carried out in part exemplarily using the object points 12, 22. Said object points 12, 22 are examples of the object points altogether, which are observable by the coordinate measuring device 10. The object space is accordingly not scanned pointwise, but rather the observable scene as a whole is captured. The object point 22 is therefore also exemplary of the body part 20, further object points of which, insofar as they are visible to the coordinate measuring device 10, are simultaneously captured.

The coordinate measuring device 10 is configured as an optical measuring system 24 that has a housing 26. A stereo camera system 28 having a first image sensor unit 30 and a second image sensor unit 32 is accommodated in the housing 26. The image sensor units 30, 32 comprise optical image sensors 34 and 36, respectively. The image sensors 34, 36 are sensitive to electromagnetic radiation that is emitted by the lighting apparatus 16 and reflected by the object point 12 and the object 18, or that is emitted by the marking element 14. Both image sensors 34, 36 have a high resolution that preferably matches.

The image sensor units 30, 32 are arranged on the coordinate measuring device 10 in known spatial orientation to each other.

The coordinate measuring device 10 comprises a data processing unit 38 that is also accommodated in the housing 26. The image sensors 34, 36 are coupled to the data processing unit 38 such that luminosity characteristics data sets, which are generable by the image sensor units 32, 34 and comprise a respective luminosity information, are transmitted to the data processing unit 38 and are processed thereby. For this purpose, an executable computer program is stored in the data processing unit 38.

The data processing unit 38 may determine the coordinates of the object points 12, 22 by triangulation of the luminosity characteristics data sets. If the object points move in the space, they may be tracked by the coordinate measuring device 10.

The drawing shows schematically how the object points 12, 22 may be seen by the first image sensor unit 30 at direction vectors 40 and 42, respectively. In corresponding manner, the object points 12, 22 are seen by the image sensor unit 32 at direction vectors 44 and 46, respectively. The accuracy with which the direction vectors 40 to 46 may be indicated using the image data sets is usually relatively high. However, in the case of stereo camera systems 28 as described hereinabove, there is the possibility that ambiguities in the evaluation of the image data sets may arise due to the position of different object points 12, 22 to each other.

To increase the accuracy with which the position of object points 12, 22 may be indicated, the coordinate measuring device 10 comprises at least one spacing measuring unit 48. The spacing measuring unit 48 is configured as a time-of-flight (TOF)-measuring unit 50 and comprises an image sensor in the form of a PMD-sensor 52. Further, the time-of-flight (TOF)-measuring unit 50 comprises an illumination unit 54 for illuminating objects to be imaged.

The PMD-sensor 52 provides at least one spacing data set, though may additionally provide a luminosity characteristics data set. The illumination unit 54 and the lighting apparatus 16 emit light of different spectral ranges.

With the TOF-measuring unit 50, the object points 12, 22 may be configured in such a way that the spacing of the object points 12, 22 is determined by the TOF-measuring unit 50. For this purpose, the illumination unit 54 illuminates the object points 12, 22. For example, the propagation time of the light from emission to detection is measured, such that, using the propagation time, a distance information may be determined with the PMD-sensor 52 in a spatially resolved manner. The TOF-measuring unit 50 may provide a spacing data set and transmit it to the data processing unit 38.

The TOF-measuring unit 50, in the use of self-luminous marking elements, is preferably synchronized therewith in order to trigger them, or vice versa.

The drawing shows exemplary distance vectors 56 and 58 from the TOF-measuring unit 50 to the object points 12 and 22, respectively. The spacing information in the distance vectors 56, 58 have an increased accuracy.

The spatial orientation of the TOF-measuring unit 50 and the image sensor units 30, 32 to each other is preferably set and preferably known to the data processing unit 38.

For determining the position of the object points 12, 22, the data processing unit 38 processes, in accordance with the invention, the first luminosity characteristics data set of the first image sensor unit 30 on the basis of at least the spacing data set of the TOF-measuring unit 50 before the triangulation. The first luminosity characteristics data set may hereby be supplemented by a spacing information that may be gathered from the spacing data set.

For the object points 12, 22 imaged by the first image sensor unit 30, the supplemented first luminosity characteristics data set therefore has an additional distance information in addition to the direction information. Beyond determining the directions to the object points 12, 22 symbolized using the direction vectors 40, 42, the data processing unit 38 recognizes in the thusly supplemented luminosity characteristics data set also the length of the direction vectors and thus the spacings of the object points 12, 22 from the first image sensor unit 30. The data processing unit 38 may derive these spacings from the spacing information of the TOF-measuring unit 50 by taking into account the relative arrangement of the TOF-measuring unit 50 and the first image sensor unit 30.

In corresponding manner, the data processing unit 38 preferably also processes the second luminosity characteristics data set of the second image sensor unit 32 before the triangulation with the spacing data set of the TOF-measuring unit 50. A spacing information which, in addition to the direction information from the direction vectors 44, 46, is included in the second image data set as the length(s) of said direction vectors 44, 46, is added to the second luminosity characteristics data set. The data processing unit 38 may determine the spacings of the object points 12, 22 from the spacing data set using the distance vectors 56, 58 and on the basis of the relative position of the TOF-measuring unit 50 and the second image sensor unit 32.

Provision may be made for the TOF-measuring unit 50 to provide, beyond the spacing data set, a luminosity characteristics data set that comprises luminosity information about the observed object points 12, 22. The data processing unit 38 may identify the image points of the object points 12, 22 in the luminosity characteristics data set with image points of the object points 12, 22 in the image data sets. This allows for creating a redundant information, so that an association of the image data set with the luminosity characteristics data sets is facilitated.

The data processing unit 38 then determines the coordinates of the object points 12, 22 in the space by triangulation. Since the respective luminosity characteristics data sets are supplemented by the spacing information from the spacing data set, a substantially more accurate determination of the object points 12, 22 than in conventional coordinate measuring devices may occur.

Ambiguities like ghost points, for example, as they may arise in conventional stereo camera systems, are mostly avoidable even before the triangulation.

The coordinate measuring device 10 comprises a storage unit 60 that may be integrated into the data processing unit 38. Attributes for observable object points may be stored in the storage unit 60. For example, the geometry of the marking apparatus may be stored with the relative positions of the marking elements to each other. In particular, the storage unit 60 comprises attributes about the anatomy of the patient, specifically about the body part 20.

In the data evaluation, the data processing unit 38 may use the attributes stored in the storage unit 60 for identifying the images of the object points in the image data sets and in the spacing data set. The attributes may be considered with respect to time upon tracking the object points.

This offers the possibility, for example, of storing 3D-matching algorithms in the data processing unit 38 and executing a tracking of the anatomy of the patient, without it hereby being necessary to mark the body part 20 with a marking apparatus. This allows for operating with less invasiveness.

Overall, surface measurements and tracking, in which the surface is captured by a close-meshed net of supporting object points, are possible with comparatively low computing power.

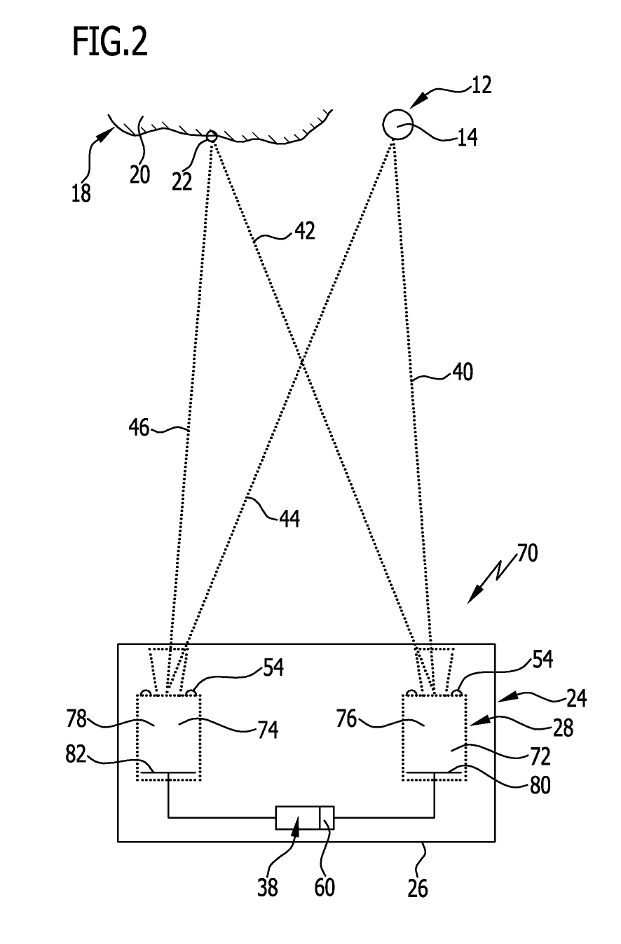

FIG. 2 shows a coordinate measuring device in accordance with the invention, which is attributed with the reference numeral 70, for carrying out a method in accordance with the invention. Identical reference numerals are used for features and components of the coordinate measuring devices 10 and 70 which are the equivalent or which have the same effect.

The coordinate measuring device 70 differs from the coordinate measuring device 10 in that, in place of the image sensor units 30, 32, which merely provide luminosity characteristics data sets, combined image sensor spacing measuring units 72, 74 are provided. These form in particular time-of-flight (TOF)-measuring units 76, 78 having PMD-sensors 80, 82. The TOF-measuring unit 50 is omitted.

The combined image sensor spacing measuring units 72, 74 are configured such that in each case one luminosity characteristics data set and one spacing data set is providable by the PMD-sensors 80 and 82, respectively. The data sets are fed to the data processing unit 38. The luminosity characteristics data set and the spacing data set of one respective combined unit 72, 74 may be evaluated by the data processing unit 38. It is hereby possible to add the spacing information from the corresponding spacing data set to each luminosity characteristics data set, in order to provide a respective three-dimensional data set. As a result, a direction vector 40, 44 and 42, 46 respectively, and at the same time the corresponding distance vector, i.e. the lengths of the direction vectors, may be determined for each object point 12, 22. Ambiguities may be ruled out, already before the triangulation by the data processing unit 38. In the triangulation, the data processing unit 38 may in this way determine the coordinates of the object points 12, 22 in the space more accurately than is the case with a conventional coordinate measuring device.

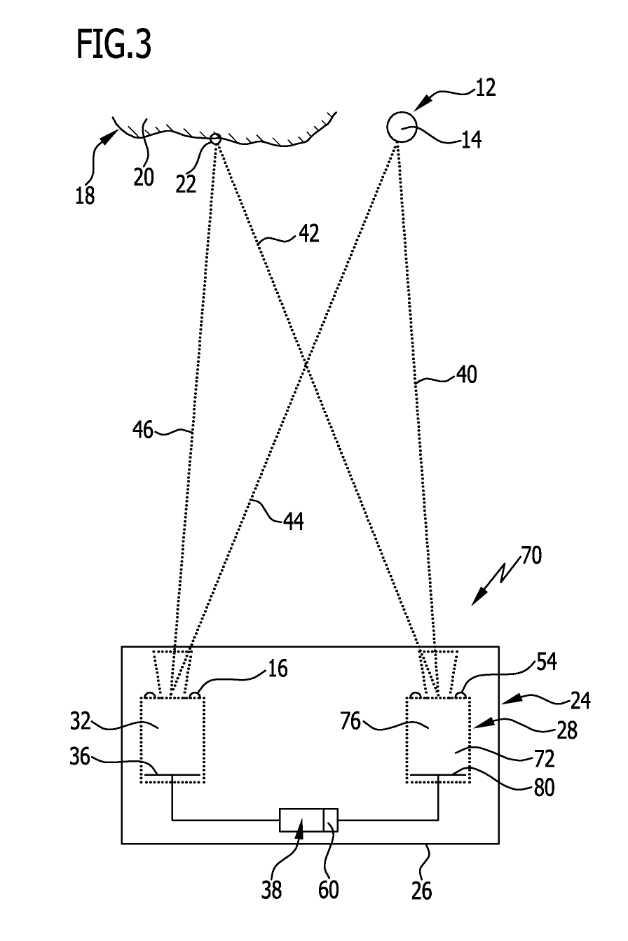

FIG. 3 shows a coordinate measuring device in accordance with the invention, attributed with the reference numeral 90, for carrying out a method in accordance with the invention. Identical reference numerals are used for features and components of the coordinate measuring device 10, 70, and 90 which are equivalent or which have the same effect.

In the coordinate measuring device 90, the combined image sensor spacing measuring unit 72 is used that forms the TOF-measuring unit 76 having the PMD-sensor 80. This provides, as in the coordinate measuring device 70, both a luminosity characteristics data set as well as a spacing characteristics data set that may be transmitted to the data processing unit 38. The data processing unit 38 may add the spacing information to the luminosity characteristics data set and create a three-dimensional data set. In addition to the information about the direction vectors 40, 42 to the object points 14, 22, a distance vector, i.e. the length of a respective direction vector, may in this way be determined at the same time. Ambiguities are able to be avoided even before the triangulation.

In addition, the image sensor unit 32 having the image sensor 36 is used in the coordinate measuring device 90. Using the luminosity characteristics data set provided by the image sensor 36, the data processing unit 38 may determine the direction vectors 44, 46 to the object points 14, 22.

Upon triangulating the first luminosity characteristics data set of the PMD-sensor 80, which is supplemented by the spacing information, with the luminosity characteristics data set of the image sensor 36, the coordinates of the object points 12, 22 in the space may be more accurately determined than in the case in a conventional coordinate measuring device.

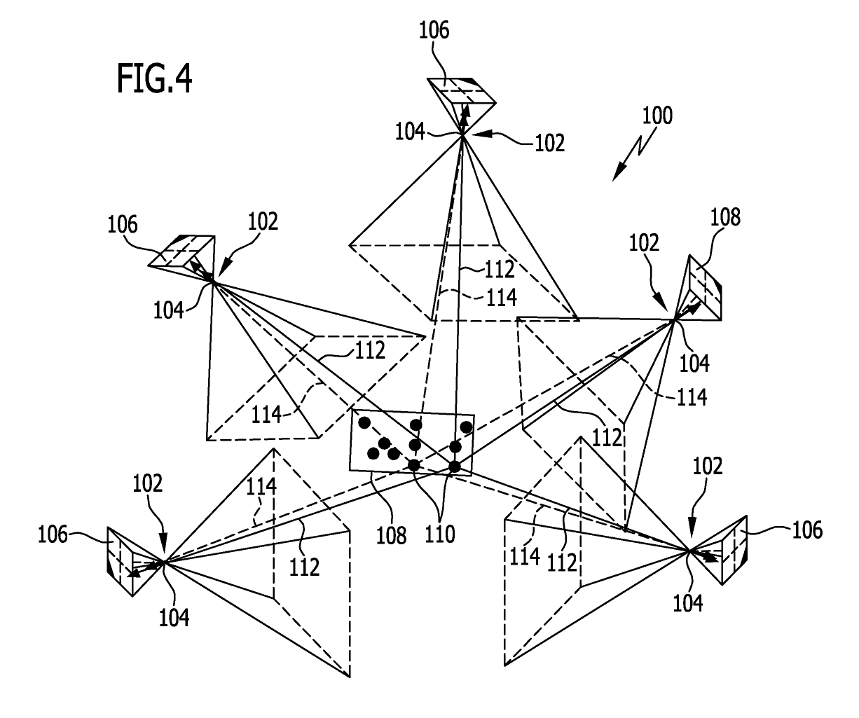

FIG. 4 shows schematically how a camera system 100, which may comprise a multitude of cameras 102, may be calibrated. The cameras 102 may be freely positionable. Presently, five cameras 102 are exemplarily shown, which may correspond to the image sensor units 30, 32, the TOF-measuring unit 50 or the combined image sensor spacing measuring units 72, 74.

FIG. 4 thereby schematically depicts a respective imaging center 104 and an image plane 106 of a camera 102, as well as an object 108 having a multitude of object points 110.

The relative orientation of the object points 110 is known to the data processing unit not shown in FIG. 4. This additional information is stored in the storage unit. By means of the additional information, the measurements from the image data may be associated with the object points 110. In the presence of at least one TOF-measuring unit, a spacing information of the object point (distance vector) as described above may also be taken into account in addition to the position of the object point (direction vector). By means of a cluster triangulation, the respective image data of the camera 102 may be simultaneously processed by the data processing unit. Ideally, the image rays 112, 114 of a respective object point 110 intersect at the same point. The imaging characteristics of the camera system 100 may be determined in this way, in particular the relative orientation of the camera 102 and the orientation of the camera 102 to the object 108.

### REFERENCE NUMERAL LIST

10 coordinate measuring device

12 object point

14 marking element

16 lighting apparatus

18 object

20 body part

22 object point

24 measuring system

26 housing

28 stereo camera system

30 first image sensor unit

32 second image sensor unit

34 image sensor

36 image sensor

38 data processing unit

40 direction vector

42 direction vector

44 direction vector

46 direction vector

48 spacing measuring unit

50 TOF-measuring unit

52 PMD-sensor

54 illumination unit

56 distance vector

58 distance vector

60 storage unit

70 coordinate measuring device

72 combined image sensor spacing measuring unit

74 combined image sensor spacing measuring unit

76 TOF-measuring unit

78 TOF-measuring unit

80 PMD-sensor

82 PMD-sensor

100 camera system

102 camera

104 imaging center

106 image plane

108 object

110 object point

112 image ray

114 image ray

What is claimed is:

1. Medical radiation-based coordinate measuring device, comprising a first image sensor unit and a second image sensor unit for providing a first luminosity characteristics data set and a second luminosity characteristics data set, respectively, and a data processing unit for determining the object coordinates of object points in the space, which are imaged by means of electromagnetic radiation, by triangulation of the luminosity characteristics data sets, wherein the coordinate measuring device comprises at least one spacing measuring unit for the object points for providing at least one spacing data set, and wherein the data processing unit is configured and programmed in such a way that it, before the triangulation, adds a spacing information to at least the first luminosity characteristics data set on the basis of a spacing data set.

2. Coordinate measuring device in accordance with claim 1, wherein the data processing unit is configured and programmed in such a way that it, before the triangulation, adds a spacing information to the second luminosity characteristics data set on the basis of a spacing data set, or wherein the data processing unit is configured and programmed in such a way that it triangulates the first luminosity characteristics data set, which is supplemented by the spacing information, with the second luminosity characteristics data set.

3. Coordinate measuring device in accordance with claim 1, wherein the at least one spacing measuring unit is or comprises a time-of-flight (TOF)-measuring unit that generates the spacing data set using a light propagation time method.

4. Coordinate measuring device in accordance with claim 3, wherein the time-of-flight (TOF)-measuring unit has an illumination unit for illuminating the object points with light of a spectral range that differs from a spectral range to which the image sensor units are sensitive.

5. Coordinate measuring device in accordance with claim 1, wherein a spacing measuring unit, which is spatially separated from the image sensor units, is provided.

6. Coordinate measuring device in accordance with claim 1, wherein the coordinate measuring device comprises at least one combined image sensor spacing measuring unit that forms an image sensor unit for providing a luminosity characteristics data set and a spacing measuring unit for providing a spacing data set, and wherein the spacing information is added to the luminosity characteristics data set on the basis of the spacing data set.

7. Coordinate measuring device in accordance with claim 1, wherein the coordinate measuring device comprises two combined image sensor spacing measuring units and wherein the respective spacing information is added to the respective luminosity characteristics data set on the basis of the respective spacing data set.

8. Coordinate measuring device in accordance with claim 1, wherein the first image sensor unit and the second image sensor unit have a joint image sensor that has two image sensor regions that each deliver a luminosity characteristics data set.

9. Coordinate measuring device in accordance with claim 1, wherein two spacing measuring units have a joint image sensor that has two image sensor regions that each deliver a spacing data set.

10. Coordinate measuring device in accordance with claim 1, wherein the resolution of optical sensors of the image sensor units and/or of spacing measuring units is identical.

11. Coordinate measuring device in accordance with claim 1, wherein two or more image sensor units and/or two or more spacing measuring units are identically configured.

12. Coordinate measuring device in accordance with claim 1, wherein the image sensor units and/or the at least one spacing measuring unit are sensitive in at least one of the following spectral ranges at least over a specifiable or specified wavelength range:
infrared;
visible light;
ultraviolet.

13. Coordinate measuring device in accordance with claim 1, comprising more than two image sensor units, wherein a luminosity characteristics data set is generable with a respective image sensor unit.

14. Coordinate measuring device in accordance with claim 1, comprising a storage unit, in which attributes of observable object points are stored, wherein the data processing unit is configured and programmed in such a way that it uses said attributes in the identification and tracking of the object points.

15. Coordinate measuring device in accordance with claim 14, wherein the attributes relate to at least one of the following:
the anatomy of a patient;
a medical instrument;
an implant.

16. Coordinate measuring device in accordance with claim 1, wherein the coordinate measuring device is configured as or comprises a measuring system having a housing accommodating the image sensor units and at least one spacing measuring unit or wherein the image sensor units and the at least one spacing measuring unit are freely positionable relative to each other.

17. Coordinate measuring device in accordance with claim 16, wherein the data processing unit is integrated into the housing, or wherein the data processing unit is positioned in a separated housing of the coordinate measuring device.

18. Medical radiation-based coordinate measuring method, in which a first luminosity characteristics data set and a second luminosity characteristics data set, respectively, are provided with a first image sensor unit and a second image sensor unit, and object coordinates of object points in the space, which are imaged by means of electromagnetic radiation, are determined with a data processing unit by triangulation of the luminosity characteristics data sets, wherein at least one spacing data set is provided with at least one spacing measuring unit, and the data processing unit, before the triangulation, adds a spacing information to at least the first image data set on the basis of the spacing data set.

19. Coordinate measuring method in accordance with claim 18, wherein a time-of-flight (TOF)-measuring unit is used as a spacing measuring unit.

20. Coordinate measuring method in accordance with claim 18, wherein at least one combined image sensor spacing measuring unit that forms an image sensor unit for providing a luminosity image data set and a spacing measuring unit for providing a spacing data set is used.
